(19) United States
(12) Patent Application Publication
(10) Pub. No.: US 2010/0268055 A1
(43) Pub. Date: Oct. 21, 2010

(54) Self-Anchoring MEMS Intrafascicular Neural Electrode

(75) Inventors: Ranu Jung, Scottsdale, AZ (US); Stephen M. Phillips, Gilbert, AZ (US); James J. Abbas, Scottsdale, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Arizona Board of Regents, a Body Corporate Acting for and on Behalf of Arizona State University, Scottsdale, AZ (US)

(21) Appl. No.: 12/669,761

(22) PCT Filed: Jul. 21, 2008
(86) PCT No.: PCT/US08/70683

§ 371 Date: Jun. 25, 2010

Related U.S. Application Data
(60) Provisional application No. 60/950,643, filed on Jul. 19, 2007.
(60) Provisional application No. 60/991,958, filed on Dec. 3, 2007.

(51) Int. Cl. A61B 5/04; C12N 13/00; H01R 43/00; A61N 1/05

(52) U.S. Cl. 600/377; 435/173.1; 216/17; 607/118

(57) ABSTRACT

The present invention provides a self anchoring electrode for recording, measuring and/or stimulating nerve activity in nerves and/or nerve fascicles of the peripheral nervous system, and methods for using such a self anchoring electrode.

### CROSS REFERENCE TO RELATED APPLICATIONS

This application claims priority to U.S. Provisional Application Ser. No. 60/950,643, filed Jul. 19, 2007, and to U.S. Provisional Application Ser. No. 60/991,958, filed Dec. 3, 2007, each of which is incorporated by reference herein in its entirety.

### STATEMENT OF GOVERNMENT INTEREST

This invention was made with government support under grant number EB003629 awarded by the National Institute of Health. The government has certain rights in the invention.

### BACKGROUND OF THE INVENTION

In recent years advances have been made in the field of neurobiology, in particular in the development of neuroprosthetic devices for motor control. An important aspect in the further advancement of the field and, for example, the control systems for neuroprosthetic devices, will be the ability to obtain stable spatiotemporally distributed recording of neural activity chronically. While the ability to record neural activity plays a critical role in developing this technology, the development of neural interfaces that can provide spatiotemporally distributed stimulation of neural tissue will also be a key to the development of neuroprosthetic devices.

Typical systems that provide electrical stimulation for neuroprosthetic devices can be applied either on the skin or directly to the nerve. The disadvantages of surface (skin) stimulation include that it is awkward to use and requires that electrodes be placed in the proper location upon every use. Additionally, large currents must be applied with these systems and in people having partial neural sensation, for example, persons with incomplete spinal cord injury, such stimulation can be painful. Implantable electrode systems could overcome some of these problems by being self-contained within the body.

Current nerve interface-based electrodes have several significant technological shortcomings. For example, glass micropipette electrodes can be used as suction electrodes to record and monitor neural activity, however these electrodes are difficult to establish and secure an adequate fit with the nerve. More commonly, nerve cuff electrodes are used to record neural activity. Nerve cuffs can attain higher signal amplitudes and decrease the amount of noise in measurement. Compared to other neural nerve interface based electrodes, the nerve cuff is relatively stable over long-term recording periods. Nevertheless, a major shortcoming of the nerve cuff electrode arises when recording data from a short segment of the nerve, because of the difficulty in placing the electrodes in confined spaces. The nerve cuff can induce changes in the tissue and is covered by connective tissue. The shape of the nerve can change when it completely fills the cuff, which can reduce neural activity over time. Another type of electrode, the Utah electrode array, which resembles a miniature bed of nails, can only be implanted transversely and does not have a self-anchoring mechanism.

Longitudinal intrafascicular electrodes (“LIFEs”) are useful for recording data from sensory fascicles in peripheral nerves, as well as for stimulating motor fascicles also in peripheral nerves. These electrodes are typically made using metallic wires (See, e.g., Dhillon & Horch, IEEE Trans. Neural Syst. Rehabil. Eng., (2005) 13:468-72; Dhillon et al., J. Nerophysiol., (2005) 93:2625-33; Li et al., Microsurgery, (2005) 25:561-65; Yoshida & Horch, IEEE Trans. Biomed. Eng., (1993) 40:492-94; Zheng et al., Microsurgery, (2008) 28:203-09). Polymer based electrodes have also been manufactured and used (See, e.g., Lawrence et al., J. Neurosci. Methods, (2003) 131:9-26; Lawrence et al., IEEE Trans. Neural Syst. Rehabil. Eng., (2004) 12:345-48; McNaughton & Horch, J. Neurosci. Methods, (1996) 70:103-110). Nevertheless, these electrodes are difficult to deploy and use because they need to be threaded through the peripheral nerve and tacked using epineural sutures at both the proximal and distal ends (See, e.g., Dhillon et al., J. Hand Surg. (Am), (2004) 29:605-18; and Malagodi et al., Ann. Biomed. Eng., (1989) 17:397-410).

Micromechanics or micro-machines, or more commonly MicroElectroMechanical Systems (MEMS) relate to technologies based on an integration of mechanical elements, such as sensors and actuators, and/or electronics on a common substrate through the utilization of micro-fabrication technology. MEMS range in size from a few microns to a few millimeters. While the electronics are typically fabricated using Integrated Circuit (IC) process sequences (e.g., CMOS, Bipolar, or BICMOS processes), micro-mechanical components are commonly fabricated using compatible “micro-machining” processes that selectively etch away parts of a substrate (e.g., a silicon wafer) or add new structural layers to form mechanical and electromechanical devices. MEMS bring together silicon-based microelectronics with micro-machining technology, thereby making possible the realization of a complete system-on-a-chip. MEMS augment the computational ability of microelectronics with the sensitivity and control capabilities of microsensors and/or microactuators. Common examples of such electrical and mechanical combinations include gyroscopes, accelerometers, micromotors, and sensors of micrometer size, all of which may need to be left free to move after encapsulation and packaging. MEMS can be used within digital to analog converters, air bag sensors, logic, memory, microcontrollers, and video controllers. Accordingly MEMS devices are used in a number of applications and industries such as military electronics, commercial electronics, automotive electronics, ink-based printers, biotechnology, and telecommunications.

In order to ensure the further advancement of neuroprosthetic devices, there is a need for new materials and approaches for recording distributed neural activity from the peripheral nervous system. MEMS technology can provide new materials, devices, and methods for recording and/or stimulating nerve activity that would vastly improve or restore limb movement in people with injuries such as spinal cord injuries, amputations, and neurological movement disorders. In addition, stimulation of other nerves such as those innervating the bladder and bowel could help restore function for controlling contraction of smooth muscle to restore impaired function.

### SUMMARY OF THE INVENTION

In one aspect, the invention provides an intrafascicular neural electrode comprising a microelectromechanical system (MEMS) comprising a stem structure, at least one barb structure attached to the stem structure, and at least one conductive trace located on the stem structure or the stem structure and the barb structure, wherein: (A) the stem structure comprises a lead end and a contact end; (B) the barb structure comprises (i) a base end in direct contact with the stem structure, (ii) an unattached distal tip end located opposite from the base end; wherein the barb structure comprises at least two layers comprising (a) a first layer; and (b) a second layer, wherein the first and the second layers have different thermal expansion coefficients, such that when the barb structure is at a first temperature it is in a first, zero stress position, and when at a second temperature it is in a second, flex position.

In another aspect, the invention provides a method for measuring and/or recording activity in a neural cell from the peripheral nervous system (PNS) comprising (I) attaching to the neural cell an electrode system comprising (1) a neural electrode comprising a microelectromechanical system (MEMS) comprising a stem structure, at least one barb structure attached to the stem structure, and at least one conductive trace located on the stem structure or the stem structure and the barb structure, wherein: (A) the stem structure comprises a lead end and a contact end; (B) the barb structure comprises (a) a base end in direct contact with the stem structure, and (b) an unattached distal tip end located opposite from the base end; wherein the barb structure comprises at least two layers comprising (i) a first layer; and (ii) a second layer, wherein the first and the second layers have different thermal expansion coefficients, such that when the barb structure is at a first temperature it is in a first, zero stress position, and when at a second temperature it is in a second, flex position; and a device that can measure neural activity in electrical communication with the neural electrode; and (II) measuring and/or reading the neural activity detected by the at least one electrode pad.

In a further aspect, the invention provides a method for stimulating a neural cell from the peripheral nervous system (PNS) comprising (I) attaching to the cell an electrode system comprising (1) a neural electrode comprising a microelectromechanical system (MEMS) comprising a stem structure, at least one barb structure attached to the stem structure, and at least one conductive trace located on the stem structure or the stem structure and the barb structure, wherein: (A) the stem structure comprises a lead end and a contact end; (B) the barb structure comprises (a) a base end in direct contact with the stem structure, and (b) an unattached distal tip end located opposite from the base end; wherein the barb structure comprises at least two layers comprising (i) a first layer; and (ii) a second layer, wherein the first and the second layers have different thermal expansion coefficients, such that when the barb structure is at a first temperature it is in a first, zero stress position, and when at a second temperature it is in a second, flex position; and (2) a device that can provide a stimulus to neural cells in electrical communication with the neural electrode of (1); and (II) applying an electric stimulus to the neural cell by inputting a stimulus to the neural cell through the at least one conductive trace.

The invention also relates to neuroprosthetic devices, methods of treating a patient, and methods of augmenting nervous system function in non-impaired persons comprising the intrafascicular neural electrodes as described herein. The invention further relates to methods for making the intrafascicular neural electrodes described herein. Further additional aspects and embodiments of the invention are described in the following detailed description of the invention.

### BRIEF DESCRIPTION OF THE DRAWINGS

FIG. 1. Cross section and plan views of non-limiting embodiments of a single trace or multi-trace intrafascicular neural electrode with active recording/stimulation trace(s) and cantilever barb anchors along the substrate stem structure.

FIG. 2. Focused Ion Beam (FIB) micrograph of top view of one embodiment of a manufactured barb anchor and a portion of a stem structure. The barb has two ends, one of which is not attached to the stem structure and is free to move (the unattached distal tip end), while the other “base” end is attached to the stem structure. The barb incorporates five conductive traces on its structure with bond pads located remotely on the stem structure.

FIG. 3. General schematic representation of a deployed self-anchoring intrafascicular neural electrode device within a nerve fascicle.

### DETAILED DESCRIPTION OF THE INVENTION

All publications, patents and patent applications cited herein are expressly incorporated by reference for all purposes. Within this application, unless otherwise stated, terms are used according to their commonly understood meanings within the relevant art. As used herein the terms “a,” “an,” and “the” are meant to include both the singular and plural forms of the terms they precede (e.g., “a cell” can refer to one or a plurality of cells).

As used herein, the term “cell,” “nerve,” or “neural cell” means at least one cell associated with the nervous system of an animal (including a human). Thus, the term “cell” or “neural cell” encompasses single cells as well as an aggregate of cells that can be part of, or associated with, a neuron or nerve, unless specifically noted otherwise. “Peripheral nerves” can contain a number of fibers of either the somatic or autonomic nervous system, and are not part of the central nervous system (CNS). A “nerve” is a bundle of nerve fibers enclosed by a nerve sheath. A “nerve fascicle” is a plurality of nerve fibers organized and bundled within the lamellated connective tissue (perineurium). A plurality of nerve fascicles can be organized within a protective sheath called the epineurium, forming the peripheral nerve.

The invention provides novel devices and methods for recording distributed neural activity in cells from the peripheral nervous system (PNS) such as, for example, mammalian spinal roots. The invention also provides novel devices and methods for stimulating neural activity in cells from the PNS including those nerve cells that control smooth muscle contraction around organs (such as the bladder and the bowels) and can help improve impaired function resulting from impaired or damaged nerve cells. The device utilizes MEMS technology and is based on a bi-material actuation mechanism that utilizes the differential in thermal expansion coefficients between various material components comprising the device which induce a physical movement of at least a part of the device as a function of temperature. The use of MEMS technology allows for manufacture of several devices on a single silicon wafer using batch processing techniques that are compatible with integrated circuit manufacturing. This enables the development of on-chip electronics for filtering, amplification and signal processing at a low manufacturing cost.

In one aspect, the invention provides an intrafascicular neural electrode comprising a microelectromechanical system (MEMS) comprising a stem structure, at least one barb structure attached to the stem structure, and at least one conductive trace located on the stem structure or the stem structure and the barb structure, wherein: (A) the stem structure comprises a lead end and a contact end; (B) the barb structure comprises a base end in direct contact with the stem structure, and an unattached distal tip end located opposite from the base end; wherein the barb structure comprises at least two layers comprising a first layer; and a second layer, wherein the first and the second layers have different thermal expansion coefficients, such that when the barb structure is at a first temperature it is in a first, zero stress position, and when at a second temperature it is in a second, flex position.

The intrafascicular neural electrodes of the invention can comprise at least one barb structure located at any point along the length of the stem structure. In one embodiment of this aspect, the electrode comprises more than one barb structure, which can each be located at a discrete point along the length of the stem structure, or at substantially the same point along on the stem structure. In certain embodiments, the electrode comprises at least two or more barb structures, which can be oriented such that when at the zero stress position the distal tip end of each barb structure is closer to the contact end of the stem structure than to the lead end of the stem structure. In other embodiments, at least one of the barb structures is oriented such that when at the zero stress position the distal tip end is closer to the contact end of the stem structure than to the lead end of the stem structure, and at least one of the barb structures is oriented such that when at the zero stress position the distal tip end is closer to the lead end of the stem structure than to the contact end of the stem structure.

The intrafascicular neural electrode comprises at least one barb structure that comprises at least two layers; a first layer and a second layer. The term “barb” is used herein to describe any type of projecting member (e.g., a beam-like structure) which comprises at least two layers that have different thermal expansion coefficients. The barb(s) is located at any position along the length of the stem structure. Certain non-limiting embodiments of barb structures (e.g., with or without other conductive trace, with various orientations relative to the stem structure, etc.) are described herein and are illustrated in the Figures. The first layer can comprise any material that is well known in the art of MEMS and/or silicon wafer manufacturing, and which is biocompatible. As noted above, the first layer has a thermal expansion coefficient that is different than the thermal expansion coefficient of the material of the second layer.

The second layer of material located on the barb structure can also comprise any material that is well known in the art of MEMS and/or silicon wafer manufacturing, and which is biocompatible, as long as it has a different thermal expansion coefficient than the first layer, defined above. In an embodiment the second layer is layered on top of, and is coextensive with, the first layer.

The first and second layers comprising the barb structure(s) of the intrafascicular neural electrode can be selected based on the intended use of the electrode, particularly based on the relevant temperature ranges typical of a given application. For example, electrodes for use in in vivo systems will comprise barbs comprising a first layer and a second layer that have different thermal expansion coefficients particularly in the range of body temperatures, such that a difference of at least 5° C. from typical body temperatures will deflect the barb position from a first zero stress position to a second flex position. In one embodiment, each particular barb structure can comprise different materials for the first layer and/or the second layer. Such barb configuration and manufacture would result in an intrafascicular neural electrode that could operate and self-anchor over a wide array of temperature ranges and potentially increase the number of applications in which it is operable.

The selection of materials for the devices is based on experience with both standardized semiconductor processing as well as material biocompatibility. In the field of semiconductor processing, stresses in the thin films that are used is well know as a problem to be avoided since it can lead to delamination of the chip layers or even curvature in the whole wafer resulting in device failure. The invention takes advantage of these stresses, enabling a mechanical actuation (deflectance) of the barbs as a function of temperature.

The key factors in determining the deflection characteristics are described in Equation 1 (below) for three layer barb structures, and are well known for two-layer barb structures (see, e.g., Timoshenko, S., “Analysis of Bi-Metal Thermostats,” J.O.S.A. & R.S.I, Vol. 11 pp. 233-255, 1925). Parameters relevant in considering barb deflection are (a) the coefficients of thermal expansion of the first and second layers; (b) the thickness of the layers; and (c) the adhesion between the layers. An added consideration for purposes of the invention is the inertness of the materials with respect to tissues and liquids in the body of the animal or human (biocompatibility). As noted herein, the thickness of the various layers are constrained by the overall diameter of the nerve fascicle, and the thickness of conductive layer (e.g., a conductive trace) allows for good signal transduction. Common electrical conducting layers (“conductive traces”) are fabricated from gold, titanium, nickel, and doped poly-silicon. A typical description of materials (and material properties) that can be used as the stem structure, as the first and second layers of the barb structure and as the conductive traces can be found in standard textbooks such as, for example, G. T. A. Kovacs, Micromachined Transducers Sourcebook, McGraw-Hill, 1998 at pg. 558. The thermal expansion coefficients for biocompatible materials that can be used in the electrode of the invention are well known in the art, and include the non-limiting examples of silicon 2.6; polysilicon, 2.33; silicon dioxide, 0.35, silicon nitride, 1.6; fused silica glass, 0.4; titanium, 8.6; (for the stem and barb structures) and (for conductive traces and electrical contacts e.g., wirebond pads) nickel, 13.0; titanium, 8.6, and gold, 14.2 (units are ppm/° C.).

In certain embodiments the self-anchoring electrode is used in in vivo applications (e.g., either recording of neural cell activity or stimulation of neural cells). One of skill in the art will be able to determine functional (and optimal) materials to use for the first and second layers of the barb structure. For example, such materials have thermal expansion coefficients that allow for deflectance from a zero stress position to a flex position within temperature ranges from about 2° C. to above about 35° C. Thus, in an embodiment the first temperature (zero stress) is from about 2° C. to about 23° C., and the second temperature (flex position) is about 28° C. or more. In other embodiments the zero stress temperature is about 2° C., 4° C., 7° C., 10° C., 12° C., 15° C., 18° C., 20° C., or 23° C. and the flex temperature is about 27° C., 28° C., 29° C., 30° C., 31° C., 32° C., 33° C., 34° C., 35° C., 36° C., 37° C., or 38° C. Where the flex temperature is designed to be about 32° C. or more, the zero stress temperature can be increased from 25° C. so long as the difference in the thermal expansion coefficients between the barb layers allows for a deflection with a temperature difference of at least 5° C.

As noted above for a barb comprising a three layered structure, one of skill in the art can derive the predicted deflection in the one or more barb structures based on Equation 1:

$$( {α}_{2} - {α}_{3} ) ( {T}_{2} - {T}_{1} ) - {E}_{1} {E}_{3} {t}_{1} {t}_{3} ( {α}_{1} - {α}_{3} ) ( {T}_{2} - {T}_{1} )$$

$$\frac{1}{ρ} = \frac{[ \begin{matrix}\frac{{t}_{1} + 2 {t}_{2} + {t}_{3}}{{E}_{1} {E}_{2} {t}_{1} {t}_{2} ( {t}_{1} + {t}_{2} ) - {E}_{2} {E}_{3} {t}_{2} {t}_{3} ( {t}_{2} + {t}_{3} )} + \\ \frac{{t}_{1} + {t}_{2}}{{E}_{1} {E}_{3} {t}_{1} {t}_{3} ( {t}_{1} + {t}_{2} ) - {E}_{3}^{2} {t}_{3}^{2} ( {t}_{2} + {t}_{3} )}\end{matrix} ]}{\begin{matrix}\frac{{E}_{1} {t}_{1}^{3} + {E}_{2} {t}_{2}^{3} + {E}_{3} {t}_{3}^{3}}{6 ( {t}_{2} + {t}_{3} )} [ \frac{{E}_{2} {t}_{2} + {E}_{3} {t}_{3}}{{E}_{2} {E}_{3} {t}_{2} {t}_{3}} ] - \\ \frac{\begin{matrix}( {E}_{1} {t}_{1}^{3} + {t}_{2} ) + {E}_{2} {t}_{2}^{3} + {E}_{3} {t}_{3}^{3} ) ( \frac{{t}_{1} + {t}_{2}}{{t}_{2} + {t}_{3}} ) + \\ 3 {E}_{3} {t}_{3} ( {t}_{1} + {t}_{2} ) ( {t}_{1} + 2 {t}_{2} + {t}_{3} )\end{matrix}}{6 {E}_{3} {t}_{3} ( {t}_{1} + {t}_{2} ) - \frac{6 {E}_{3}^{2} {t}_{3}^{2}}{{E}_{1} {t}_{1}} ( {t}_{2} + {t}_{3} )}\end{matrix}} - \frac{\begin{matrix}\begin{matrix}( {E}_{1} {t}_{1}^{3} + {t}_{2} ) + {E}_{2} {t}_{2}^{3} + {E}_{3} {t}_{3}^{3} ) \\ ( \frac{{t}_{1} + 2 {t}_{2} + {t}_{3}}{{t}_{2} + {t}_{3}} ) +\end{matrix} \\ 3 {E}_{3} {t}_{3} ( {t}_{1} + {t}_{2} ) {( {t}_{1} + 2 {t}_{2} + {t}_{3} )}^{2}\end{matrix}}{\begin{matrix}6 {E}_{2} {t}_{2} ( {t}_{1} + {t}_{2} ) - \\ \frac{6 {E}_{2} {E}_{3} {t}_{2} {t}_{3}}{{E}_{1} {t}_{1}} ( {t}_{2} + {t}_{3} )\end{matrix}} + \frac{( {t}_{2} + {t}_{3} )}{2}$$

wherein ρ is the radius of curvature, αᵢ, is the coefficient of thermal expansion, T is the temperature, tᵢ is the thickness of individual layer, Eᵢ is the Young's modulus, where the subscript “i” denotes the particular layer.

In an embodiment a temperature difference of about 5° C. effectively deflects the barb structure. “Effectively deflects” means that the temperature difference is adequate to induce enough deflection in the barb structure to bring the barb in contact with at least a portion of the neural cell. In other embodiments, the barb structure is designed so that a difference of about 5° C., 10° C., 15° C., 20° C., 25° C., or 30° C. or more in temperature generates enough deflection in the barb to exert enough force to “self anchor” onto a neural cell structure, for example, nerve fascicle(s) and allows the conductive trace(s) adequate contact with the neural cells for recording, measuring, and/or stimulating neural cell activity. Such force can range widely depending on the cell type, usually from about 0.01-1.0 pN, but in any case the force should not be so great as to damage the cell or the particular neural cell structure. Designing the electrode by using materials that will allow the barb to effectively deflect with a temperature difference of at least 5° C. (or more) allows for an adequate window of temperature difference such that the device can function properly in in vivo situations when small variations in body temperature exist.

The intrafascicular electrode (i.e., the stem structure and/or the barb structure) can comprise additional layers that do not have an effect on the deflectance of the barb structures, but are useful in manufacture, and include for example adhesion layers or insulating layers. Typical materials that can be used in insulating layers include silicon dioxide, undoped polysilicon, undoped silicon, silicon nitride, glass, polymers, other dielectric materials, and the like that can provide electrical insulation. In an embodiment, the material used for an insulating layer is able to maintain its insulating properties when formed into thin layers. Particularly when applied to the barb structure, the insulating layer is of a thickness such that it does not influence the deflection of the barb structure, which can be determined by one of skill in the art, but ranges typically from about 0.01-0.11 μm for barbs of about 1.3 μm thickness. Adhesion layers (e.g., a chromium layer) can be located between two layers in order to provide better adherence between the two layers than would exist in the absence of the adhesion layer. Like the insulating layer, when incorporated in the barb structure the adhesion layer is of a thickness such that it does not contribute to the thermal deflection mechanism of the barb structure.

The stem structure of the intrafascicular electrode relates to the larger, main portion of the electrode and comprises at least one conductive trace and has attached to it at least one barb structure. Together, the stem structure, the at least one barb structure, and the at least one conductive trace comprise the structure of the self-anchoring electrode. Merely for purposes of orientation, the stem structure has two ends, a “lead end” and a “contact end,” and while the contact end typically serves as the point of electrical contact of the electrode to a device that can record, measure, or stimulate neural cell activity, such contact(s) can be made at any point along the stem structure. The stem structure can comprise any type of material commonly used in MEMS and semiconductor manufacturing (e.g., silicon, polysilicon, silicon dioxide, silicon nitride, titanium, fused silica glass, etc.) so long as it has adequate biocompatibility. Unlike the barb structure, the stem structure does not deflect with a difference in temperatures. In some embodiments the stem structure comprises the same layers as the barb structure. In such embodiments, the stem comprises at least one additional layer that prevents the stem from deflecting under different temperature conditions. Such additional layers can comprise, for example, (1) a substrate such as glass or silicon of a thickness that prevents the stem structure from deflecting under different temperature conditions; (2) a top layer and a bottom layer of the same material, or a top layer and a bottom layer of materials that have substantially the same coefficients of thermal expansion (e.g., a sandwich type of layering) of a thickness that prevents the stem structure from deflecting under different temperature conditions; (3) an additional layer having a thickness and coefficient of thermal expansion designed to offset the amount of deflection under certain temperature conditions; or other methods that will be apparent to one of skill in the art.

As used herein, the term “conductive trace” describes a structure located on any portion of the stem structure or the stem structure and the barb structure that allows for electrical information to transmit in either direction between the self-anchoring electrode and a neural cell (e.g., a nerve) such that it can detect or transmit electrical impulses. For certain applications it can be advantageous for the intrafascicular neural electrode to comprise one or more conductive trace(s) located along substantially the entire length of the stem structure. For intrafascicular neural electrodes comprising more than one barb structure, one or more conductive trace(s) can be located on each barb structure, on several of the barb structures, only on a single barb structure, or on none of the barb structures. The conductive traces can be put in electrical communication to any type of device that can record, measure, and/or stimulate activity in a neural cell, by any known method in the art such as, for example, connection through bond pads located on the stem structure. The material comprising the conductive trace can be any material that is suitable for use in an electrode that is known in the art, such as the non-limiting examples of metals, including gold, platinum, copper, silver, aluminum, nickel, titanium, doped polysilicon, and the like. In one embodiment, the conductive trace comprises a biocompatible material, such as gold or platinum. The conductive trace can be a continuous layer of material, or a plurality of traces can be patterned discretely as to form independent conductive traces. In one embodiment, the intrafascicular electrode comprises a plurality of spatially distributed conductive traces. Accordingly, when the electrode comprises a pattern of discrete conductive traces (which in certain embodiments may be referred to herein as “more than conductive trace”), the regions can be interconnected with each other and to an input or measuring device, or they can be connected independently to put them in electrical communication with at least one input or measuring device.

The dimensions and configuration of the intrafascicular neural electrode can be tailored optimally depending on the application for which it will be used (i.e., whether it functions to record nerve cell activity, stimulate nerve cell activity, and/or used as part of a neuroprosthetic, etc.) as well as the source and type of nerve in which it will be used. Typically, intrafascicular neural electrodes of the invention have dimensions that allow it to fit within the nerve fascicle, without causing damage to the cell. For example, the stem structure is typically on the order of tens of microns in width, for example about 10, 20, 30, 40, 50, 60, 70, 80, or 90 microns. The approximate maximum practical stem width is about 200 microns (e.g., about 100, 110, 120, 130, 140, 150, 160, 170, 180, 190, or 200 microns), depending on the type and source of nerve tissue in which the electrode will be used. In certain embodiments, the stem is about 25-150 microns in width, and the barb width is less than 25 to less than 150 microns.

The width of the one or more barb structures can vary widely, and can be of a width up to about the width of the stem structure. One of skill will recognize that the amount of deflection in a barb structure (i.e., the difference in position from zero stress position to flex position) is largely determined by the length to thickness ratio of the barb structure. A barb structure will typically have a length to thickness ratio of at least about 10:1, to about 200:1 (e.g., 10:1, 20:1, 30:1, 40:1, 50:1, 60:1, 70:1, 80:1, 90:1, 100:1, 110:1, 120:1, 130:1, 140:1, 150:1, 160:1, 170:1, 180:1, 190:1, or 200:1). Typically, the length to thickness ratio is about 100:1.

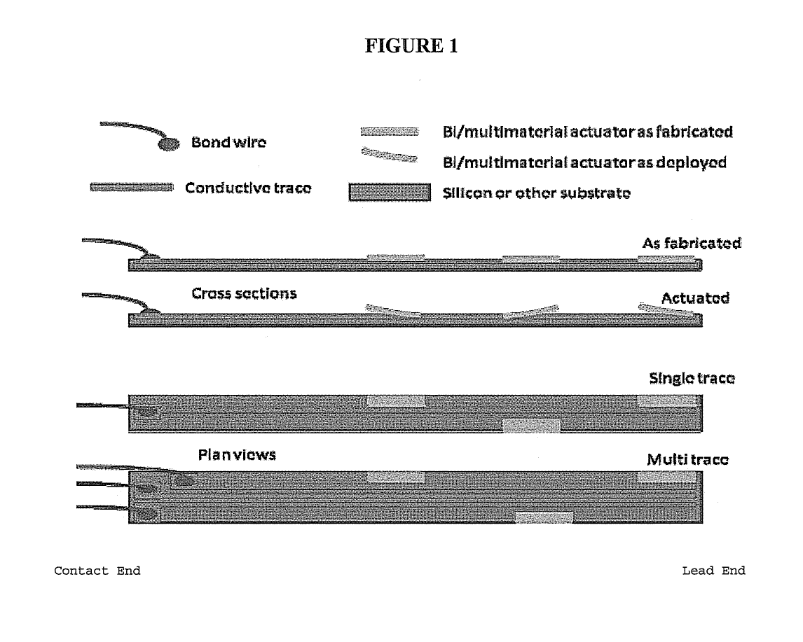

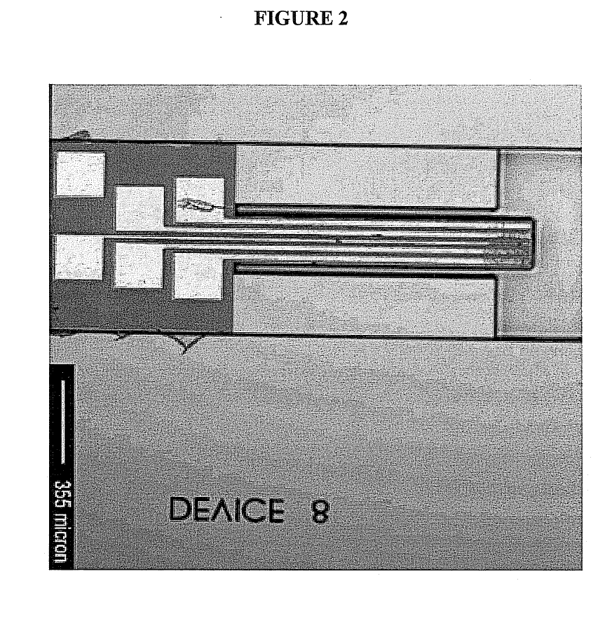

The geometry of the intrafascicular neural electrode allows for it to be inserted within the nerve fascicles of a nerve. In one embodiment the electrode (i.e., the stem structure and the one or more barb structures) is wire-like in shape and dimension (i.e., long length and thin diameter). In another embodiment the electrode (i.e., the stem structure and the one or more barb structures) are board-like in shape and dimension (i.e., long length and relatively thin width and thickness (see, e.g., FIG. 1-2).

As noted above, the electrodes can comprise more than one barb structure attached to the stem structure, such as 2, 3, 4, 5, 6, 7, 8, 9, 10, or more barb structures. Each individual barb structure can independently comprise one or more conductive traces or electrode pads, such as 2, 3, 4, 5, or more electrode pads, or the barb structure(s) can comprise no electrode pads.

The conductive trace(s) or electrode pad(s) in the individual barb structure, or the conductive trace(s) or electrode pad(s) in the entire intrafascicular electrode can be configured such that they each detect or transmit electrical stimuli independently from one another, or such that they operate as a single unit (linked in “series” with a single input/measurement device).

As will be understood by those of skill in the art, the electrode and its various component elements can be made by any process well known in the art of MEMS and/or micro-electrode manufacture. Some non-limiting manufacturing methods include those disclosed in Madou, M., Fundamentals of Microfabrication, CRC Press 1997, ISBN 0-8493-9451-1; Kovacs, G., Micromachined Transducers Sourcebook, McGraw-Hill 1998, ISBN 0-0729-0722-3; Senturia, S. D., Microsystem Design, Kluwer 2000, ISBN 0-7923-7246-8; and Sze, S. M. ed., Semiconductor Sensors, Wiley 1994, ISBN 0-4715-4609-7.

In one aspect, the invention provides a method of making an intrafascicular electrode comprising:
(a) providing a substrate;
(b) layering a first material having a first thermal expansion coefficient onto the substrate;
(c) layering a second material having a second thermal expansion coefficient onto the layer of the first material;
(d) applying a mask to the layer of the second material to form a pattern for a conductive layer;
(e) applying a conductive layer on top of the masked layer of the second material, forming a conductive trace;
(f) applying a second mask to the surface of the material generated in (e) to form the dimensions of a barb structure having a base end, a distal tip end and two adjacent sides;
(g) etching along three sides of the barb structure defined by the mask of (f) to form openings along the barb structure, creating a distal tip end, and two adjacent sides;
   wherein

the distal tip end of the barb structure is not attached to the substrate; and

the first thermal expansion coefficient and the second thermal expansion coefficient are not the same.

In another aspect, the invention provides methods for measuring and/or recording activity in a neural cell from the peripheral nervous system (PNS) comprising

(A) attaching to the neural cell an electrode system comprising
(1) an intrafascicular neural electrode comprising a microelectromechanical system (MEMS) comprising a stem structure, at least one barb structure attached to the stem structure, and at least one conductive trace located on either the stem structure or the barb structure or both, wherein:
   (i) the stem structure comprises:
      (a) a lead end and a contact end
   (ii) the barb structure comprises:
      (a) a base end in direct contact with the stem structure;
      (b) an unattached distal tip end located opposite from the base end;
   wherein the barb structure comprises at least two layers comprising:
      (1) a first layer; and
      (2) a second layer;
   wherein the first and the second layers have different thermal expansion coefficients, such that when the barb structure is at a first temperature it is in a first, zero stress position, and when at a second temperature it is in a second, flex position; and
(2) a device that can measure neural activity in electrical communication with the intrafascicular neural electrode of (1); and

(B) measuring and/or recording the neural activity detected by the at least one conductive trace.

In a further aspect, the invention provides methods for stimulating one or a plurality of neural cells from the peripheral nervous system (PNS) comprising:

(A) attaching to the neural cells an electrode system comprising
(1) an intrafascicular neural electrode comprising a microelectromechanical system (MEMS) comprising a stem structure, at least one barb structure attached to the stem structure, and at least one conductive trace located on either the stem structure or the barb structure or both, wherein:
   (i) the stem structure comprises:
      (a) a lead end and a contact end
   (ii) the barb structure comprises:
      (a) a base end in direct contact with the stem structure;
      (b) an unattached distal tip end located opposite from the base end;
   wherein the barb structure comprises at least two layers comprising:
      (1) a first layer; and
      (2) a second layer;
   wherein the first and the second layers have different thermal expansion coefficients, such that when the barb structure is at a first temperature it is in a first, zero stress position, and when at a second temperature it is in a second, flex position; and
(2) a device that can provide a stimulus to neural cells in electrical communication with the intrafascicular neural electrode of (1); and

(B) applying an electric stimulus to the neural cell by inputting a stimuli to the neural cell through the at least one conductive trace.

In these above methods of the invention, the electrodes are used to interface with the peripheral nervous system in order to record neural activity and/or stimulate neurons by detecting or providing either electrical or chemical stimuli to or from the neural cell(s) via the electrode. The electrode systems include devices that are in electrical communication with the intrafascicular neural electrode. The electrical communication between the device and the electrode can be established before, during, or after implantation of the electrode in the nerve. Methods for attachment are well known in the art (e.g., direct attachment through contacts (e.g., wires attached to wirebond pads), wireless communication, and the like). Further the measuring device can be incorporated on the electrode structure (an “on-board” measuring device).

In an aspect the invention provides a method treating a condition or disorder associated with impaired neural function in a patient comprising the intrafascicular electrode of the invention, wherein the condition or disorder associated with impaired neural function is selected from impairment or loss of tactile sensation; impaired hearing; impaired vision; impaired motor control; impaired bladder control; Parkinson's disease; paraplegia, tetraplegia; amyotrophic lateral sclerosis; loss of bowel control; erectile dysfunction; loss of cognitive function; gastroparesis, irregular heartbeat; and pain.

Thus, the electrode of the invention is useful for providing renewed neural function or sensation to a patient who has previously lost neural function or sensation such as, for example, in the case of an amputation. The electrode can provide sensory information to the patient by stimulating the sensory afferent nerves in order to relay information from one or more sensors mounted on a prosthesis to measure touch, temperature, force, position, orientation, and the like. Another use of the electrode is to record the activity of motor neurons and use the recorded signal to drive the motors on a prosthesis. Yet another use would be combining the electrode with a separate sensor in a closed loop system, wherein the separate sensor is used to measure a physiological variable such as, for example, end tidal carbon dioxide during respiration, and use that variable to trigger stimulation of the phrenic nerve via the electrode. In yet further applications, the electrode can be implanted in the spinal cord or brain, and upon deployment via self-anchoring, the electrode can be designed to release a pharmaceutical such as, for example, nerve growth factor, by and number of mechanisms known to one of skill in the art (e.g., by degradation of a slow release coating, or release of active agent upon electrical impulse supplied to the electrode.).

In an embodiment of this aspect, one or more surfaces of the electrode can be doped with at least one chemical agent that is released upon electrical stimulation. Thus, step (B) above would initiate a release of the at least one chemical agent upon applying an electric stimulus. Such chemical agents can be any agent that can modulate cellular activity, such as inhibiting one or more cellular activity or inducing one or more cellular activity. In this embodiment, chemical agents should be selected for compatibility with the component materials of the intrafascicular neural electrode, and should be present in amounts effective to modulate cellular activity.

Devices that can provide either a stimulus to a neural cell or record and/or measure neural cell activity (or both) are well known in the art, for example, stimulators and recording amplifiers such as those available from CWE, Inc. (Ardmore, Pa.); World Precision Instruments, Inc. (“WPI Inc.”; Sarasota, Fla.); A-M Systems, Inc. (Sequim, Wash.); and Neuralynx, Inc. (Bozeman, Mont.). Such devices can be connected to one or more of the intrafascicular neural electrode(s) of the invention by any connection that allows the device to receive and input data from and to the one or more electrode(s). Custom stimulators and neural recording devices that can be implanted within the body or optionally located outside of the body can also be utilized.

In another aspect, the invention relates to neuroprosthetic devices that comprise one or more of the intrafascicular electrodes of the invention. This aspect of the invention broadly relates to any type of neuroprosthetic device that can be designed and used to replace or improve the function of an impaired nervous system or to augment the function of a non-impaired nervous system. Prosthetic devices that incorporate a system for sending and/or receiving electrical stimulus to neural cells are known generally in the art, and can be modified to work with the intrafascicular electrodes disclosed herein (e.g., cochlear implants, brain and brainstem implants (e.g., auditory, visual cortex (vision), motor (movement control), etc.), spinal and lumbar anterior root implants, implants that support function of the autonomous nervous system such as, for example, bladder control (sacral anterior root stimulator), and the like).

Sensory/Motor prosthetics seek to establish an interface with neurons that provide limb movement and touch sensation (for example, an implant interfaced directly into the median nerve fibres for movement of, and recording of touch feedback in, an artificial limb. Warwick, K., et al., Archives of Neurology, (2003); 60(10):1369-1373). Several strategies exist that seek to achieve advanced control of neural prosthetics, in general. Direct chronic brain implants record neuronal signals from the motor cortex, while methods such as electroencephalography (EEG) and functional magnetic resonance imaging (fMRI) obtain motor commands non-invasively (Polikov, V. S., et al., J Neurosci Methods (2005 Oct. 15); 148(1):1-18; Schwartz, A. B., et al., Neuron (2006 Oct. 5); 52(1):205-20). The recorded signals are decoded into electrical signals, and input into assistive devices or motorized prosthetics. Traditional myoelectric prostheses utilize surface electromyography (EMG) signals from the remains of the amputated limb (Sears, H. H., Atlas of Limb Prosthetics, (1992); “Trends in upper-extremity prosthetics development”). For example, a patient may flex a shoulder muscle in order to generate EMG signals that may be used to send “bend elbow” command to the prosthesis. Targeted reinnervation is another surgical method which makes use of the patient's existing nerves and is aimed to provide an amputee with improved control over motorized prosthetic devices and to regain sensory feedback. (Kuiken T., Phys Med Rehabil Clin N Am., (2006, February); 17(1):1-13). Such motor neuroprosthetics find use in a wide variety of patient class, particularly those patients that have a disease or condition that impairs their ability to control muscle function, movement, and/or communicate (e.g., amputees, persons with bladder control problems, Parkinson's disease, tetraplagia, amyotrophic lateral sclerosis, and the like). The sacral anterior root stimulator has been used to improve bladder emptying in patients that have lost the ability to control bladder function (e.g., because of paraplegia caused by spinal cord injury or lesion(s)), and have been shown to assist with controlling defecation and sustaining male erection.

Visual prosthetics are typically targeted and implanted within the visual cortex area of the brain, and can improve vision in patients having significantly impaired vision (but not total blindness). Auditory prosthetics such as the cochlear implant and the auditory brain stem implant are surgically implanted into the cochlea, or brainstem, of patients who are deaf or severely hard of hearing. These implants are typically coupled with external components including a microphone, speech processor, and transmitter.

Pain relief prosthetics such as the Spinal Cord Stimulator or (Dorsal Column Stimulator) are used to treat chronic neurological pain. Typically these implants are set near the dorsal surface of the spinal cord and an electric impulse generated by the device provides a “tingling” sensation that alters the perception of pain by the patient. In one type of implant arrangement, a pulse generator or RF receiver is implanted remotely (e.g., in the abdomen or buttocks) from the lead/electrode, which is connected to the generator by a wire harness.

Cognitive prosthetics (e.g., hippocampal prosthesis) are aimed at restoring cognitive function by replacing circuits within the brain damaged by stroke, trauma or disease. Berger, T., et al. IEEE Engineering in Medicine and Biology Magazine, (2005; September/October):30-46 “Restoring Lost Cognitive Function”; and Graham-Rowe, D., New Scientist, (2003); 12 March “World's first brain prosthesis revealed”).

In an embodiment of this aspect of the invention, a neuroprosthetic device incorporates the intrafascicular electrode of the invention and is designed to induce or control a physiological response in a subject. Several non-limiting examples of devices that can incorporate the electrodes of the invention include stimulators such as those for pacemakers for the vagus nerve (e.g., from Cyberonics, Inc., Houston, Tex.), for the pudendal nerve (bladder control), the ENTERRA™ Therapy subsystem (Medtronic, Inc., Minneapolis, Minn.) for stimulating the stomach muscles and treatment of gastroparesis, for deep brain stimulation (e.g. from Medtronic, Inc., Minneapolis, Minn.), and for pain relief (e.g. from Medtronic, Inc., Minneapolis, Minn.).

In a related aspect, the invention provides a method of augmenting neurological function in a person with normal neurological function comprising connecting to the peripheral nervous system of the person at least one intrafascicular electrode of in invention, and providing a stimulus via the intrafascicular electrode, wherein the stimulus elicits sensations in the sensory nerves of the peripheral nervous system. In an embodiment, this method of augmenting neurological function in a person with normal neurological function comprises connecting to the peripheral nervous system of the person at least one intrafascicular electrode of the invention, recording neural activity from the peripheral nervous system, transmitting the recorded neural activity to an external device, wherein the transmission of recorded activity generates a response in the external device; and providing a return stimulus from the external device via the intrafascicular electrode, wherein the return stimulus elicits sensations in the sensory nerves of the peripheral nervous system of the person.

This aspect of the invention relates to virtual reality applications, such as remote control of a robot or exoskeleton by a person wherein the recording of neural activity in the person can be transmitted to the remotely located robot or exoskeleton and control its movement. Sensors on the robot or exoskeleton can provide a return stimulus to the nervous system of the person, providing a sensation to the person that can relate to the position, pressure, or temperature, or the like at the location of the robot. Persons involved in a wide range of professions can benefit from such augmentation of neurorlogical function such as, for example, surgeons, aircraft pilots, bomb squad technicians, hazardous materials (hazmat) teams, crane operators, and demolition experts.

### EXAMPLES

### Example 1

### Design and Manufacture of a Prototype Intrafascicular Neural Electrode

Standard MEMS processes are used to construct a series of the neural electrodes of the invention, such as wafer level batch microfabrication methods described in Madou, M., Fundamentals of Microfabrication, CRC Press 1997, ISBN 0-8493-9451-1; Kovacs, G., Micromachined Transducers Sourcebook, McGraw-Hill 1998, ISBN 0-0729-0722-3; Senturia, S. D., Microsystem Design, Kluwer 2000, ISBN 0-7923-7246-8; and Sze, S. M. ed., Semiconductor Sensors, Wiley 1994, ISBN 0-4715-4609-7. Known processes employing oxidation, structural polysilicon growth and metal deposition are combined to generate the stem and barb self-anchoring configuration, as well as the location and design of the conductive traces. A standard single crystal silicon wafer having dimensions of 0.5 mm (thickness) and 100 mm (length) allows for the production of several thousand electrodes of the invention. Depending on the desired dimension of the electrode, the substrate can be thinned using any method known in the art such as, for example, lapping, chemo-mechanical polishing, or fabrication on a silicon-on-insulator (SOI) substrate. The in-plane device dimensions are varied across the wafer in order to produce electrode devices varying in length, width, and conductive trace configuration(s) that are dependent on the intended use of the particular electrode.

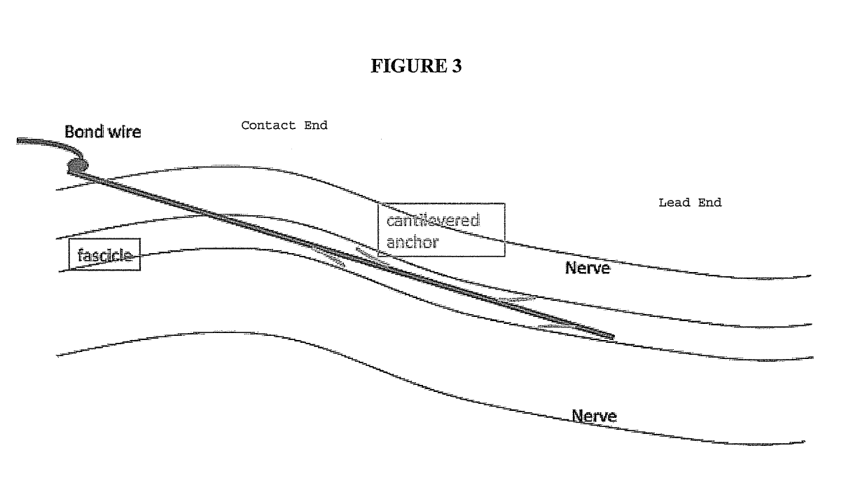

An intrafascicular electrode structure such as depicted in FIG. 3 was manufactured utilizing an autoCAD designed mask for patterning, and a Heidelberg DWL66 Laser Writermask-making tool that was used to apply the positive photoresist, producing chrome masks on glass substrate. Briefly, the device processing begins with cleaning the substrate wafer in buffered oxide etchant (BOE) (20:1 ammonium fluoride (NH₄F) buffer and hydrofluoric acid (HF) etchant). A 500 Å layer of silicon dioxide was grown, on top of which polysilicon was deposited (0.4 μm) in a polysilicon furnace (Tempress Lindberg). The polysilicon layer was patterned with a first mask to allow for subsequent gold deposition. A Plasmalab M80 deep reactive ion etcher (RIE) was used for patterning both silicon dioxide and polysilicon layers. A layer of chromium (˜10 nm) was deposited on the polysilicon by thermal evaporation (Edward II thermal evaporator) which serves as an adhesion layer for the gold. A gold layer having a thickness of about 0.8 μm, was applied by electroplating and patterned with a second mask. The conductive gold layer serves as the conductive traces and signal paths for neural recording or stimulation. These gold conductive traces are connected to wirebond pads located on the stem portion of the structure. An additional bond pad was included for electrical reference in the same patterned gold layer. After patterning with a third mask, a 0.1 μm layer of silicon dioxide was deposited using a PlasmaQuest RPCVD as a top layer to provide electrical isolation over all regions, except on the bond pads. Another 10 nm layer of chromium layer was been used between the gold paths and this top silicon dioxide layer for increased adhesion. The device was patterned with a fourth mask and a surface technology systems (STS) deep-silicon etcher was used to make the opening along the barb so that the barb can be released, and to allow eventual singulation if the individual beam malfunctions. The resulting structure in depicted in FIG. 3.

### Example 2

### Characterization of the Thermal and Mechanical Self-Anchoring System

A set of intrafascicular electrodes having a wide variation in dimensional parameters, number and configuration of barb structures, and number and location of conductive traces are analyzed to determine deflections and forces as a function of temperature for the variously configured electrodes. A controlled temperature deionized water bath, followed by a saline bath determines the device “deflectance temperature” within the 5°-30° C. range. The displacements induced by the temperature change are measured using optical microscopy and optical interferometry. The force calculations are determined from the materials properties (modulus) that are extracted form non-device portions of the processed wafers and the dimensions observed by optical microscopy.

### Example 3

### Self-Anchoring Evaluation Using Animal Nerve Tissue

The self-anchoring capabilities of the intrafascicular neural electrodes are tested using neural tissue derived from animals. American bullfrogs are anesthetized in a bath of 1-3% MS-222 solution for about 20-30 min. The anesthetized frogs are decerebrated, the spinal cord is pithed, and the hindleg peripheral nerves from are removed. The peripheral nerve(s) are stretched to various tensions so that performance of the self-anchoring mechanism is characterized based on device placement under given tensions.

Based on the results from the anchoring and performance studies using frog nerve tissue, experiments are designed using rodent nerve tissue. Both fixed and fresh samples of rat (female Long-Evans) nerve tissue from the phrenic, sciatic, and tibial nerves, as well as lumbosacral nerve roots are used to develop optimized operating conditions and device design.

### Example 4

### Electrical Recording and Stimulation In-Vivo

Neural activity is recorded from spinal nerve roots and peripheral nerves in anesthetized rodents, in order to determine an effective arrangement of the conductive traces on the device as well as optimal placement of the electrode. From the ventral side, the C5 cervical spinal roots are exposed on both the left and right side. These spinal roots for the phrenic nerve that innervates the diaphragm, allowing for the recording of autonomous neural respiratory activity. The intrafascicular electrodes are stored in cooled saline (5-10° C.) so that the barb structures are in the “zero stress” position (“undeployed”). The nerve root is continuously irrigated with cold saline solution until the intrafascicular electrode device has been positioned appropriately, as visualized through a dissecting microscope or other visualization instrument. Partial epineural dissections allow for fascicular visualization over a distance of approximately 1 cm or more. The lead end of the electrode is inserted into the fascicle and moved longitudinally along the fascicle until the entire electrode device is within the fascicle. Once the electrode is positioned, the irrigation is halted, and the area surrounding the spinal root is dried. Once the temperature of the electrode device warms to body temperature, it self-anchors to the nerve root. Multiple electrodes (e.g., from 2, 3, 4, 5, 6, 7, 8, 9, or 10 or more) can be implanted within multiple fascicles.

Neural activity is continuously recorded with the aid of filtering and amplification of the signal (AM systems, 100 Hz-3 KHz). The data is also sampled and recorded in real-time and a real-time power spectrum is displayed (Labview, National Instruments PCI-6070E). The neural activity and the power spectrum are monitored to determine whether appropriate contact and anchoring is established. All recorded data is saved for additional off-line analysis. For example wavelet analysis is used to obtain an energy profile of the neural signal, and allows for separation of the signal power into different frequency bands in non-stationary signals. The effects of different anchoring forces and electrode configurations are examined as a function of changes in the shape of the energy profile.

The ability to record neural activity simultaneously from multiple intrafascicular electrode devices are tested on additional animal subjects. These animals are ventilated and their chest cavity is opened ventrally, lateral to the sternum. Both phrenic nerves are exposed and 2 to 4 MEMS electrode devices are placed on each of the right and left phrenic nerves. The data is recorded, measured, and analyzed as described above in order to optimize electrode configuration and materials, number of electrode attachments, and recording parameters.

While the invention has been described above in terms of general aspects, certain embodiments and specific examples, the foregoing disclosure should not be taken as limiting the scope of the invention, which is defined by the following claims.

We claim:

1. An intrafascicular neural electrode comprising a microelectromechanical system (MEMS) comprising a stem structure, at least one barb structure attached to the stem structure, and at least one conductive trace located on the stem structure or the stem structure and the barb structure, wherein:
(A) the stem structure comprises:
(i) a lead end and a contact end
(B) the barb structure comprises:
(i) a base end in direct contact with the stem structure;
(ii) an unattached distal tip end located opposite from the base end;
wherein the barb structure comprises at least two layers comprising:
(a) a first layer; and
(b) a second layer;
wherein the first and the second layers have different thermal expansion coefficients, such that when the barb structure is at a first temperature it is in a first, zero stress position, and when at a second temperature it is in a second, flex position.

2. The intrafascicular neural electrode of claim 1, wherein the at least one conductive trace is located on the stem structure and the barb structure.

3. The intrafascicular neural electrode of claim 1, comprising more that one conductive trace located on the stem structure and the barb structure.

4. The intrafascicular neural electrode of claim 1, wherein the conductive trace is selected from gold, platinum, copper, silver, aluminum, nickel, titanium, or doped polysilicon.

5. The intrafascicular neural electrode of claim 1, wherein the at least one conductive trace is located along substantially the entire length of the stem structure.

6. The intrafascicular neural electrode of claim 1, comprising more than one barb structure.

7. The intrafascicular neural electrode of claim 6, comprising more than one conductive trace located along the length of the stem structure.

8. The intrafascicular neural electrode of claim 1, wherein the first layer and second layer of the barb structure are independently selected from silicon, polysilicon, silicon dioxide, silicon nitride, fused silica glass, and titanium, wherein the first layer and the second layer are not the same.

9. The intrafascicular neural electrode of claim 6, wherein the more than one barb structures are located at different discrete locations along the length of the stem structure.

10. The intrafascicular neural electrode of claim 6, wherein the more than one barb structures are attached at substantially the same location on the stem structure.

11. The intrafascicular neural electrode of claim 6, wherein the more than one barb structures are oriented such that when at the zero stress position the distal tip end of each barb structure is closer to the contact end of the stem structure than to the lead end of the stem structure.

12. The intrafascicular neural electrode of claim 6, wherein at least one of the more than one barb structures is oriented such that when at the zero stress position the distal tip end is closer to the contact end of the stem structure than to the lead end of the stem structure, and at least one of the more than one barb structures is oriented such that when at the zero stress position the distal tip end is closer to the lead end of the stem structure than to the contact end of the stem structure.

13. The intrafascicular neural electrode of claim 1, wherein the stem is about 25-150 microns in width, and the barb width is less than the width of the stem.

14. The intrafascicular neural electrode of claim 1, wherein the second temperature is about 28° C. or higher.

15. The intrafascicular neural electrode of claim 1, wherein the first temperature is about 2° C. to about 23° C.

16. The intrafascicular neural electrode of claim 1, wherein the different thermal expansion coefficients are selected such that an increase of about 5° C. from initial temperature positions the barb structure in the flex position.

17. A method for measuring and/or recording activity in a neural cell from the peripheral nervous system (PNS) comprising
(I) attaching to the neural cell an electrode system comprising
(1) a neural electrode comprising a microelectromechanical system (MEMS) comprising a stem structure, at least one barb structure attached to the stem structure, and at least one conductive trace located on either the stem structure or the barb structure or both, wherein:
(A) the stem structure comprises:
(a) a lead end and a contact end
(B) the barb structure comprises:
(a) a base end in direct contact with the stem structure;
(b) an unattached distal tip end located opposite from the base end;
wherein the barb structure comprises at least two layers comprising:
(i) a first layer; and
(ii) a second layer;
wherein the first and the second layers have different thermal expansion coefficients, such that when the barb structure is at a first temperature it is in a first, zero stress position, and when at a second temperature it is in a second, flex position; and
(2) a device that can measure neural activity in electrical communication with the neural electrode of (1); and
(II) measuring and/or recording the neural activity detected by the at least one conductive trace.

18. A method for stimulating a neural cell from the peripheral nervous system (PNS) comprising:
(I) attaching to the neural cells an electrode system comprising
(1) a neural electrode comprising a microelectromechanical system (MEMS) comprising a stem structure, at least one barb structure attached to the stem structure, and at least one conductive trace located on either the stem structure or the barb structure or both, wherein:
(A) the stem structure comprises:
(a) a lead end and a contact end
(B) the barb structure comprises:
(a) a base end in direct contact with the stem structure;
(b) an unattached distal tip end located opposite from the base end;
wherein the barb structure comprises at least two layers comprising:
(i) a first layer; and
(ii) a second layer;
wherein the first and the second layers have different thermal expansion coefficients, such that when the barb structure is at a first temperature it is in a first, zero stress position, and when at a second temperature it is in a second, flex position; and
(2) a device that can provide a stimulus to neural cells in electrical communication with the neural electrode of (1); and
(II) applying an electric stimulus to the neural cell by inputting a stimuli to the neural cell through at least one conductive trace.

19. A neuroprosthetic device comprising the neural electrode of claim 1.

20. A method of treating a condition or disorder associated with impaired neural function in a patient comprising connecting to the peripheral nervous system of patient at least one intrafascicular electrode of claim 1, wherein the condition or disorder associated with impaired neural function is selected from impairment or loss of tactile sensation; impaired hearing; impaired vision; impaired motor control; impaired bladder control; Parkinson's disease; paraplegia, tetraplegia; amyotrophic lateral sclerosis; loss of bowel control; erectile dysfunction; loss of cognitive function; gastroparesis, irregular heartbeat; impaired respiration, and pain.

21. A method of augmenting neurological function in a person with normal neurological function comprising connecting to the peripheral nervous system of the person at least one intrafascicular electrode of claim 1, and providing a stimulus via the intrafascicular electrode, wherein the stimulus elicits sensations in the sensory nerves of the peripheral nervous system of the person.

22. A method of augmenting neurological function in a person with normal neurological function comprising connecting to the peripheral nervous system of the person at least one intrafascicular electrode of claim 1, recording neural activity from the peripheral nervous system, transmitting the recorded neural activity to an external device, wherein the transmission of recorded activity generates a response in the external device; and providing a return stimulus from the external device via the intrafascicular electrode, wherein the return stimulus elicits sensations in the sensory nerves of the peripheral nervous system of the person.

23. A method of making an intrafascicular electrode comprising:
(a) providing a substrate;
(b) layering a first material having a first thermal expansion coefficient onto the substrate;
(c) layering a second material having a second thermal expansion coefficient onto the layer of the first material;
(d) applying a mask to the layer of the second material to form a pattern for a conductive layer;
(e) applying a conductive layer on top of the masked layer of the second material, forming a conductive trace;
(f) applying a second mask to the surface of the material generated in (e) to form the dimensions of a barb structure having a base end, a distal tip end and two adjacent sides;
(g) etching along three sides of the barb structure defined by the mask of (f) to form openings along the barb structure, creating a distal tip end, and two adjacent sides;
wherein
the distal tip end of the barb structure is not attached to the substrate; and
the first thermal expansion coefficient and the second thermal expansion coefficient are not the same.
